(19) United States
(12) Patent Application Publication
(10) Pub. No.: US 2026/0193613 A1
(43) Pub. Date: Jul. 9, 2026

(54) 2-DIMENSIONAL CULTURE METHOD OF EMBRYOID BODIES FOR MESENCHYMAL STEM CELL DIFFERENTIATION

(71) Applicant: YEDITEPE UNIVERSITESI, Istanbul (TR)

(72) Inventors: Aysegul DOGAN, Istanbul (TR); Derya SAGRAC, Istanbul (TR); Selinay SENKAL, Istanbul (TR); Taha Bartu HAYAL, Istanbul (TR); Fikrettin SAHIN, Istanbul (TR)

(73) Assignee: YEDITEPE UNIVERSITESI, Istanbul (TR)

(21) Appl. No.: 18/577,312

(22) PCT Filed: Jun. 30, 2022
(86) PCT No.: PCT/TR2022/050684

§ 371 Date: Jan. 8, 2024

(30) Foreign Application Priority Data
Jul. 8, 2021 (TR) 2021/011151

(51) Int. Cl. C12N 5/0775

(52) U.S. Cl. CPC C12N 5/0662; C12N 2501/115; C12N 2501/727; C12N 2506/45; C12N 2533/54

(57) ABSTRACT

A method for mesenchymal stem cell differentiation from human pluripotent stem cells is provided, particularly including the step of adaptation of embryoid bodies from 3-dimensional culture to 2-dimensional culture. The method enables rapid mesoderm and mesenchymal stem cell transformation by transferring from 3-dimensional culture to 2-dimensional culture. Also, the method relates to obtaining mesenchymal stem cell, which can be used in clinical applications, with this method.

### CROSS REFERENCE TO THE RELATED APPLICATIONS

This application is the national phase entry of International Application No. PCT/TR2022/050684, filed on Jun. 30, 2022, which is based upon and claims priority to Turkish Patent Application No. 2021/011151, filed on Jul. 8, 2021, the entire contents of which are incorporated herein by reference.

### TECHNICAL FIELD

The present invention relates to a method for mesenchymal stem cell differentiation from human pluripotent stem cells, particularly comprising the step of 2-dimensional culture of embryoid bodies. Moreover, the present invention relates to obtaining mesenchymal stem cell, which can be used in clinical applications, with this method.

### BACKGROUND

Stem cells are master cells which can differentiate into the various cells that constitute the tissues of an organism and are often referred to as undifferentiated cells before transforming into other cells. Mesenchymal stem cells are of the types of adult stem cells. Adult stem cells can be classified as multipotent, oligopotent and unipotent stem cells depending on their differentiation ability. Adult stem cells are available as mesenchymal stem cells (MSCs) or hematopoietic stem cells (HSCs). Pluripotent stem cells are classified as embryonic stem cells and induced pluripotent stem cells and can differentiate into many types of cells thanks to their pluripotent properties. Several attempts have been made to use pluripotent human stem cells, which can differentiate into all cells, as a stem cell therapy product.

The obtaining, characterization, culture, and differentiation of pluripotent stem cells are crucial for stem cell studies. Development of protocols that can be applied clinically will constitute the basis of stem cell therapy-based applications. Inefficient differentiation of pluripotent stem cells in culture hinders derivation of clinically relevant terminally differentiated cells

[symbol]

It is known that mesenchymal stem cells can differentiate into cells of mesodermal origin such as chondrocyte, osteoblast, adipocyte and myoblast cells. They are a potential cell source for cell-based therapies in clinical applications, especially due to their ability to differentiate into the cells that constitute the tissues in which they are present and their unique ability of self-renewal.

In the literature, it is known that human pluripotent stem cells are generally used to produce mesenchymal stem cells to solve problems in the method of culturing mesenchymal stem cell. Differentiation from human pluripotent stem cells into mesenchymal stem cells generally requires an induction procedure. Also, there are challenges such as the mesenchymal stem cells produced by these methods preserving their basic state and their low production efficiency. For these reasons, there are limitations to the use of mesenchymal stem cells as ideal cell therapy products in the fields of regenerative medicine and cell therapy.

Inability to obtain sufficient number of mesenchymal stem cells, the use of invasive methods to reach stem cells, heterogeneity observed due to factors such as health status between different tissues and individuals and age, rapid aging during proliferation in culture and tissue incompatibility observed in allogeneic therapies are the problems observed in mesenchymal stem cell culture and therapies.

Obtaining pluripotent stem cell-derived mesenchymal stem cells will be able to eliminate these problems. The protocols used for obtaining pluripotent stem cell-derived mesenchymal stem cells requires optimization. Performing the process of obtaining pluripotent stem cell-derived mesenchymal stem cells in culture in a short time and by using inexpensive techniques are the problems that need to be solved to obtain cells that can be applied in therapy. For this reason, establishing protocols of obtaining pluripotent stem cell-derived mesenchymal stem cell, and keeping the obtained cells in culture by preserving their stem cell properties are important and necessary for treatment protocols.

### SUMMARY

The present invention relates to a method for mesenchymal stem cell differentiation, which overcomes all the aforementioned problems and brings additional advantages to the related technical field.

The main objective of the present invention relates to an effective differentiation method for obtaining cells from pluripotent stem cells that are safe for clinical use.

The present invention relates to a method for mesenchymal stem cell differentiation comprising the step of forming 3-dimensional embryoid body from pluripotent stem cells in a cost-effective and efficient manner.

The objective of the present invention is to differentiate cells by transitioning from 3-dimensional culture to 2-dimensional culture, by mimicking their embryonic developmental stages and mesenchymal stem cell culture conditions. Thus, effective mesenchymal stem cell differentiation is achieved.

The present invention also includes the study for finding the optimal surface for the transition and differentiation from a 3-dimensional culture to a 2-dimensional culture. Thus, a method of effective differentiation from pluripotent stem cells has been established.

With the present invention, by implementing the transition process from 3-dimensional culture to 2-dimensional culture, the time to obtain mesenchymal stem cells has been accelerated and the cost of culture medium has been reduced.

With the present invention, it will be possible to obtain bankable mesenchymal stem cells from pluripotent stem cells.

### BRIEF DESCRIPTION OF THE DRAWINGS

The “2-Dimensional Culture Method of Embryoid Bodies for Mesenchymal Stem Cell Differentiation”, which was performed for achieving the objective of the present invention, is illustrated in the accompanying drawings, in which:

FIG. 1 illustrates the technique used to form the embryoid body. 96-well PCR plates made of polypropylene plastic material were used to form embryoid body. The bodies were obtained at the end of day two.

FIGS. 2A-2F illustrate the study related to the determination of the appropriate cell number for the formation of embryoid bodies. Embryoid body formation and area/size determination were performed with different cell numbers. At the end of the analyses, the appropriate cell number was determined as 4000 cells/well. In FIG. 2A, morphological appearance of embryoid bodies is obtained using 1×10³ cells. In FIG. 2B, morphological appearance of embryoid bodies is obtained using 2×10³ cells. In FIG. 2C, morphological appearance of embryoid bodies is obtained using 3×10³ cells. In FIG. 2D, morphological appearance of embryoid bodies is obtained using 4×10³ cells. In FIG. 2E, area calculation of embryoid bodies is obtained using different cell numbers (1×10³/2×10³/3×10³/4×10³). In FIG. 2F, diameter calculation of embryoid bodies is obtained using different cell numbers (1×10³/2×10³/3×10³/4×10³))

FIGS. 3A-3C illustrate the study related to determination of the suitable surface on which embryoid bodies will be transferred. It was determined by embryoid body diameter and area analysis that embryoid bodies preferred Matrigel-gelatin mixture for optimal attachment, proliferation and migration. FIG. 3A shows the morphological analysis of the behavior of embryoid bodies on 4 different surfaces (Gelatin, Laminin, Matrigel, Matrigel-Gelatin) on day three and six. FIG. 3B shows the diameter measurement of embryoid bodies based on surface attachment and cell migration on day three to day six. FIG. 3C shows the surface area of embryoid bodies based on surface attachment and cell migration on day three to day six.

FIG. 4 shows time-dependent illustration of the steps of the method of the present invention. Obtaining and culturing 3-dimensional embryoid bodies and differentiation protocol in 2-dimensional culture.

FIGS. 5A-5E are the illustration of mesoderm differentiation and mesoderm marker analysis of embryoid bodies. FIG. 5A shows the culture of embryoid bodies on matrigel-gelatin mixture, FIGS. 5B-SD show the time-dependent cell migration analysis, where FIG. 5B shows the illustration of the area and intensity measurements of total, core, and migration regions of embryoid bodies using color-temperature scale, FIG. 5C shows the graph of migration area measurement, FIG. 5D shows the graph of migration intensity measurement), FIG. 5E shows the analysis of mesoderm marker proteins.

FIGS. 6A-6D illustrate the mesenchymal stem cells obtained at the end of the inventive steps on day 24 and their characterization. FIG. 6A shows the fibroblast-like morphology, FIG. 6B shows the colony forming potential, FIG. 6C shows the bone differentiation, FIG. 6D shows the cartilage differentiation.

The components shown in the figures are each given reference numbers as follows:
PCR-P. 96 well PCR plate
Centrf. 4000 cell/well N2B27+bBFG+ROCKi Centrifuge
PBS. Phosphate buffer saline
TCD. 150 mm tissue culture dish
N2B27+CHIR. N2B27 medium and CHIR
DMEM+bBFG+ROCKi. Complete DMEM+bBFG+ROCKi
DMEM. Complete DMEM
Migr. Migration analysis Mesoderm differentiation analysis
Trnsf. Transfer to 10 cm tissue culture dishes
Trasf-1M. Transfer to 10 cm tissue culture dishes 1 million cell/dish
E-MSC-s. Early MSC state
MSC-s. MSC state
EBT. Embryoid body transfer
CsfEB. Cells spreading from Embryoid body

### DETAILED DESCRIPTION OF THE EMBODIMENTS

Within the scope of the present invention, pluripotent stem cells are effectively differentiated into mesoderm and mesenchymal stem cells by transferring from 3-dimensional culture to 2-dimensional culture. This allows for effective cell differentiation to occur as a protocol for mimicking the true embryonic development.

Furthermore, within the scope of the invention, it is aimed to establish a differentiation protocol with an effective method without the need for any device or instrument and an additional auxiliary substance other than those known. This method, which will be crucial for stem cell therapy research, will be a priority for stem cell therapies to be used clinically.

The present invention comprises mesenchymal stem cells produced by this method, cell therapy products containing mesenchymal stem cells, and a standardized and optimized culturing protocol, which can be used in clinical applications, for producing mesenchymal stem cells from human pluripotent stem cells.

As used herein, the term “stem cells” refers to master cells that can be renewed unlimitedly to form the cells of tissues and organs. Stem cells are totipotent, multipotent, pluripotent, oligopotent or unipotent cells.

As used herein, the term “pluripotent stem cells” refers to pluripotent stem cells that can differentiate into all three germ layers that constitute a living body, and examples of these include embryonic stem cells and induced pluripotent stem (iPS) cells.

As used herein, the term “differentiation” refers to a process by which cells become specialized in terms of structure or function during cell division, proliferation, and growth, whether in embryonic development or in the adult individual.

As used herein, the term “mesenchymal stem cell” refers to adult stem cells present in different tissues in the adult individual and to mesenchymal stem cells differentiated from the mesoderm germ layer using pluripotent stem cells.

As used herein, the term “embryoid body” refers to an aggregate formed by inducing the differentiation of pluripotent stem cells. Embryoid bodies are formed when pluripotent stem cells are cultured as a suspension in serum-free medium and are 3-dimensional structures that can differentiate into 3 germ layers.

The present invention comprises the formation of embryoid bodies from human pluripotent stem cells and the formation of mesenchymal stem cells from these bodies.

With the method developed within the scope of the invention, it is aimed to model cell migration of 3-dimensional embryoid bodies in 2-dimensional culture under in vitro conditions and to establish in vitro differentiation protocol.

The present invention relates to a method for producing mesenchymal stem cells using human pluripotent stem cells, comprising the following steps:
e) forming embryoid bodies from human pluripotent stem cells;
f) attaching embryoid bodies to a surface to enable transfer from 3-dimensional culture to 2-dimensional culture;
g) inducing embryoid bodies to allow their differentiation into mesenchymal stem cells;
h) mesenchymal stem cells preserving their mesenchymal stem cell properties and their capacity for continuous proliferation while maintaining their existence.

In step (a) of the method of the present invention, embryoid bodies are formed from human pluripotent stem cells. At this stage, human pluripotent stem cells are cultured under conditions suitable for forming embryoid body. To form said embryoid bodies, human pluripotent stem cells were first precipitated (200×g, 5 minutes) as suspension in body formation medium and cultured at 37° C. for 2 days. In the said invention, the medium referred to as the body formation medium comprises N2B27 (DMEM/F12/Neurobasal medium (1:1), Bovine Serum Albumin (BSA), N2 supplement, B27 supplement, penicillin (10.000 U/ml)-streptomycin (10.000 μg/ml), L-glutamine), ROCKi (Rho-associated protein kinase (ROCK) inhibitor), and bFGF (basic fibroblast growth factor) substances. The use of ROCKi positively affects the body formation, and humidity was provided in the environment by adding PBS and evaporation was slowed down. In this way, bodies are successfully obtained. Thereby, a method different from the existing technique for embryoid body formation was developed.

In step (b) of the method of the present invention, the embryoid bodies are attached to a surface. This step allows the transfer of embryoid bodies from 3-dimensional culture to 2-dimensional culture. For the process of attaching said embryoid bodies to the surface, the suitable surface on which the cells can migrate, and the cell number were determined. In the preferred embodiment of the invention, the surface used is a surface coated with matrigel-gelatin (1:200, 70 μg/ml matrigel-0.1% Gelatin). The embryoid bodies are placed on the coated surfaces. These bodies were placed on the coated surfaces and the migration of cells was analyzed and the results showed that the best attachment and migration performance of embryoid bodies was obtained on the surface of matrigel-gelatin mixture. Matrigel-gelatin (1:200, 70 μg/ml matrigel-0.1% Gelatin) mixture was determined as the most suitable surface for the transfer of embryoid bodies from 3-dimensional culture to 2-dimensional culture. Thus, after culturing 3-dimensional embryoid bodies in 2-dimensional form and thus accelerating the transition process from epithelial to mesenchymal, it is ensured that the obtaining mesenchymal stem cells, which will attach to the plastic surface, is accelerated.

In the said step (b), the process of attaching the embryoid bodies to a surface is carried out at the end of day two.

In step (c) of the method of the present invention, mesodermal cells are induced to enable the differentiation of embryoid bodies into mesenchymal stem cells. After being kept for 2 days, the embryoid bodies obtained in step (a) are transferred to the surface coated with matrigel-gelatin mixture (culture dish) in the presence of N2B27 medium and 3 μM CHIR (CHIR99021, a stimulant that increases the differentiation potential of mesenchymal stem cells). On day three, mesoderm marker (Aplnr, Flk-1, PDGFRα) analyses were performed on embryoid bodies and their mesoderm layer protein expressions were observed. Cells were cultured in the presence of CHIR and mesoderm differentiation was completed.

In the step (d) of the method of the present invention, it is enabled to perform continuous proliferative culture of mesenchymal stem cells while maintaining the presence of mesenchymal stem cells. At this stage, differentiation and culturing of mesenchymal stem cells are performed in the presence of a medium specific to mesenchymal stem cells to create a largely homogeneous population of mesenchymal stem cells. As of day six, cells were cultured in DMEM (1 g/L glucose), 10% FBS, 1% PSA and 5 ng/ml bFGF and in a dish containing matrigel-gelatin coated surface. Cells at day ten indicate the early mesenchymal stem cell stage and cells at day 24 indicate the adult mesenchymal stem cell stage.

Mesenchymal stem cells produced by the method of the present invention can be used directly in therapy.

In the preferred embodiment of the invention, mesenchymal stem cells produced by the method of the present invention can be differentiated into adipocytes, osteocytes, chondrocytes, epithelial, endothelial and myocytes, neurocytes and cardiomyocytes.

In addition, the present invention comprises the use of mesenchymal stem cells obtained by the method of the present invention in cell therapy products. Specifically, the said cell therapy products can be used for the formation of adipocytes, osteocytes, chondrocytes, myocytes, neurocytes and cardiomyocytes and their differentiation into various cells according to the environments.

The following examples are to better illustrate the subject matter of the invention and the subject matter of the invention is not limited to these examples.

### EXAMPLES

### Example 1—Forming Embryoid Bodies

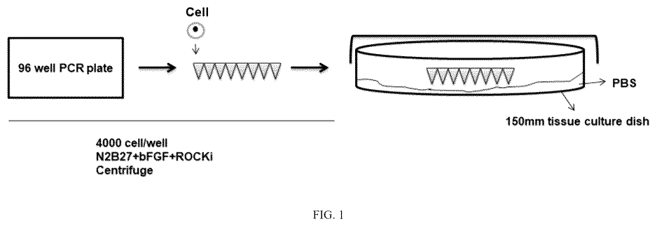

3. In the study, pluripotent stem cells were prepared in embryoid body formation medium (N2B27+Rocki (10 μM)+12 ng/ml bFGF) in 96 well plates at 1000, 2000, 3000 and 4000 cells/well in order to determine the appropriate cell number for the purpose of forming embryoid body.
4. After the cells were placed in a sterilized polypropylene plastic well PCR plate by being exposed to UV light for 30 min and precipitated at 200×g for 5 minutes, they were incubated at 37° C. for 2 days (FIG. 1).

### Example 2—Analysis of the Suitable Surface for the Transfer of Embryoid Bodies From 3-Dimensional Culture to 2-Dimensional Culture

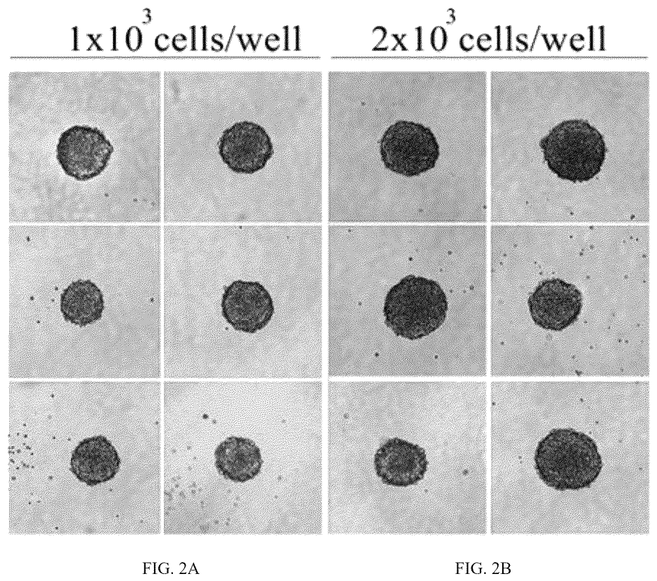

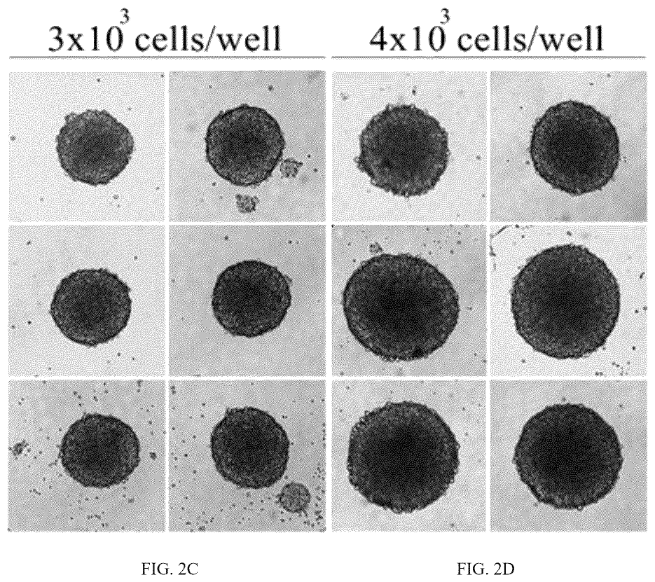

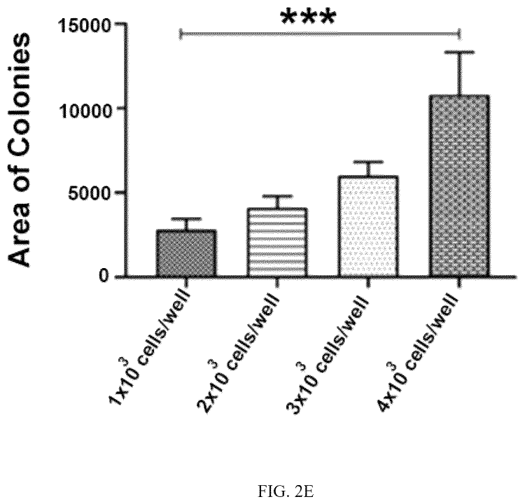

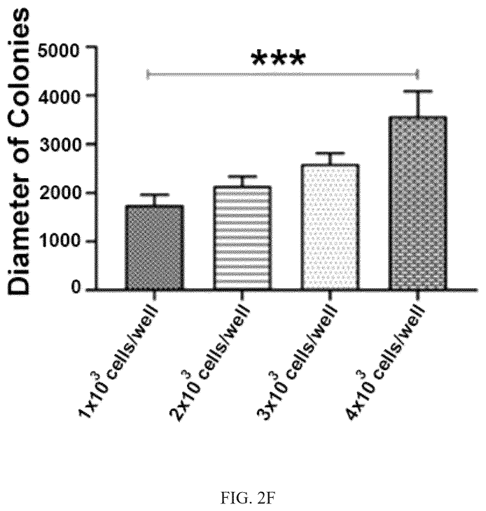

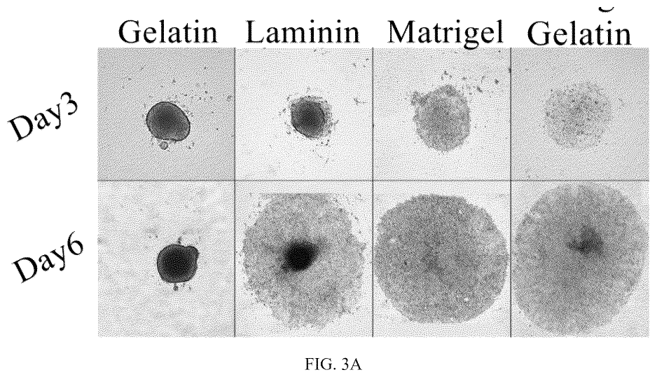

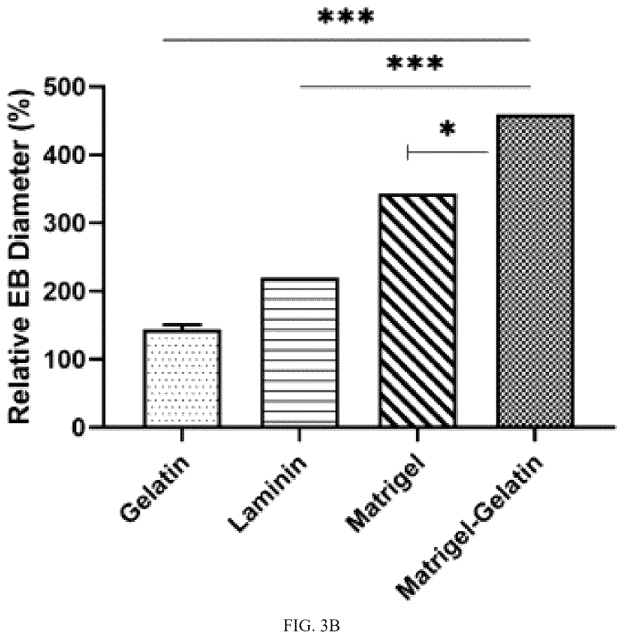

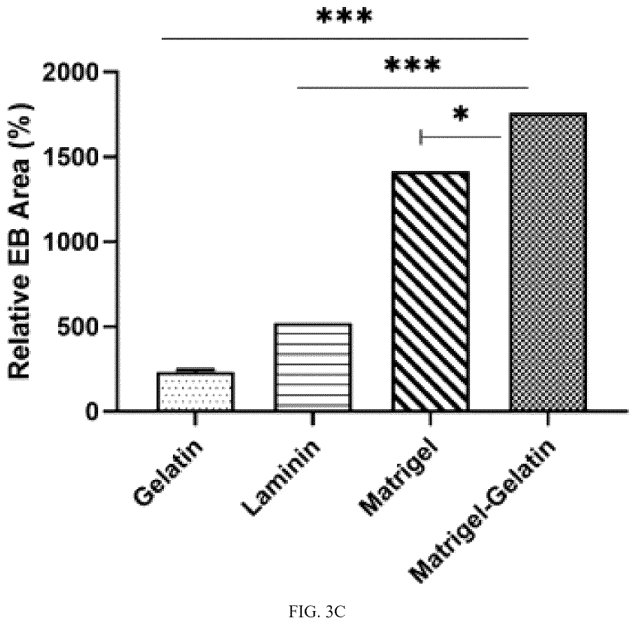

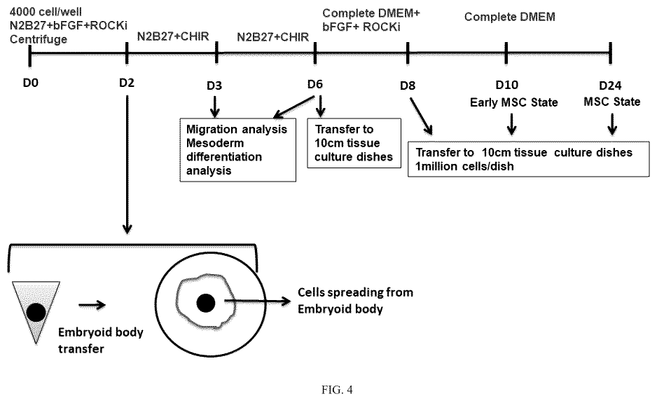

5. On day three, the embryoid bodies were transferred to 12-well culture dishes and photographed every day. Since the appropriate embryonic body formation was observed in the 4000 cells/well group by measuring the area and diameter of the formed bodies, this number of cells was used for the rest of the experiments (FIGS. 2A-2F).
6. In order to determine the suitable surface on which the cells can migrate, embryoid bodies were placed on surfaces coated with Gelatin (0.1%), 20 ng/ml Laminin, 70 μg/ml matrigel and matrigel-gelatin (1:200, 70 μg/ml matrigel-0.1% Gelatin) and the suitable surface determination analysis was performed. 12 ng/ml bFGF was added in N2B27 medium (DMEMF12/Neurobasal medium (1:1), Bovine Serum Albumin (BSA), N2 supplement, B27 supplement, penicillin (10.000 U/ml)-streptomycin (10.000 g/ml), L-glutamine) at 4000 cells/well and embryoid bodies were formed using 96-well PCR plates. These bodies were placed on different coated surfaces and the migration of cells was analyzed (FIGS. 3A-3C). According to these results, the best attachment and migration performance of embryoid bodies was obtained on the surface of matrigel-gelatin mixture.
7. Matrigel-gelatin (1:200, 70 μg/ml matrigel-0.1% Gelatin) mixture was determined as the most suitable surface for the analysis of the suitable surface for the transfer of embryoid bodies from 3-dimensional culture to 2-dimensional culture. In this way, the suitable coating surface was determined to establish a novel differentiation protocol from pluripotent stem cells.
8. Matrigel-gelatin (1:200, 70 mg/ml matrigel-0.1% Gelatin) coating is performed by adding matrigel at a ratio of 1:200 to a 0.1% Gelatin solution prepared in PBS (such that the final matrigel concentration will be 70 μg/ml matrigel) and by leaving this solution in culture dishes and keeping them at 37° C. for 1 hour. Culture dishes coated with the prepared solution can be stored and used stably for 2 weeks at 4° C. The culture dishes, which are coated and made ready-to-use, can be used even if they are kept at 37° C. for 1 week.

### Example 3—Cell Migration Analyses

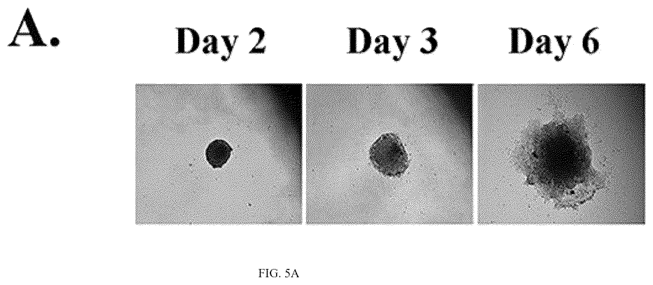

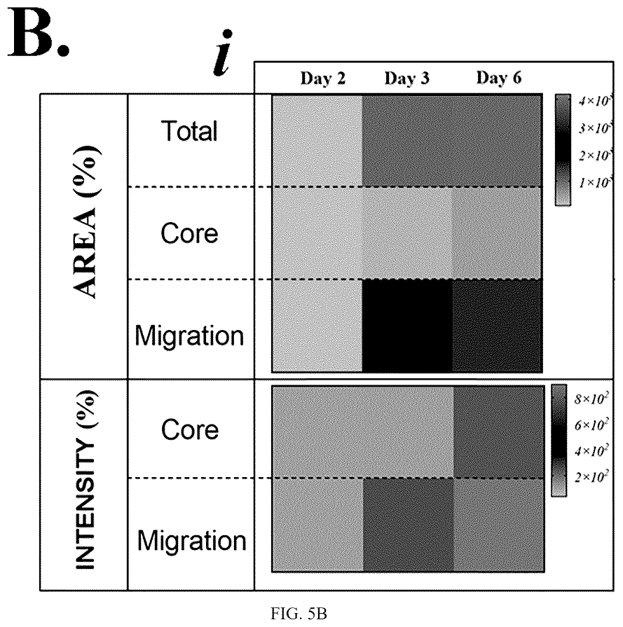

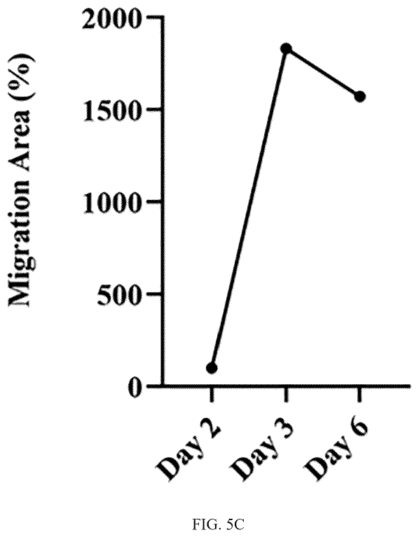

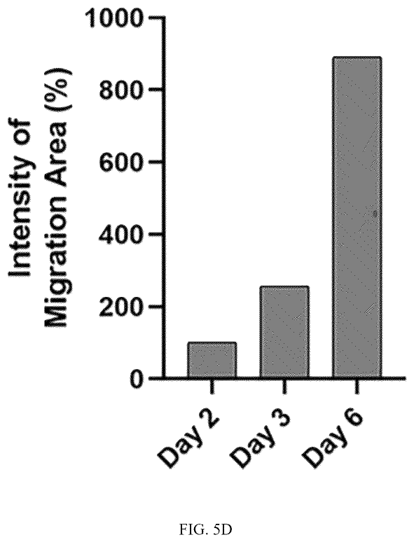

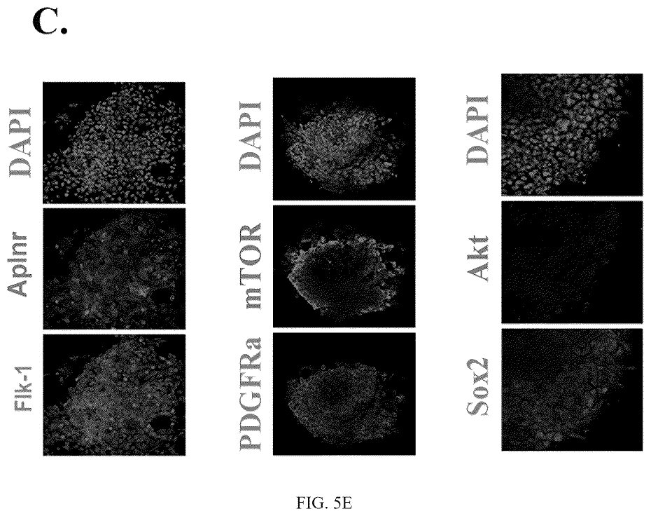

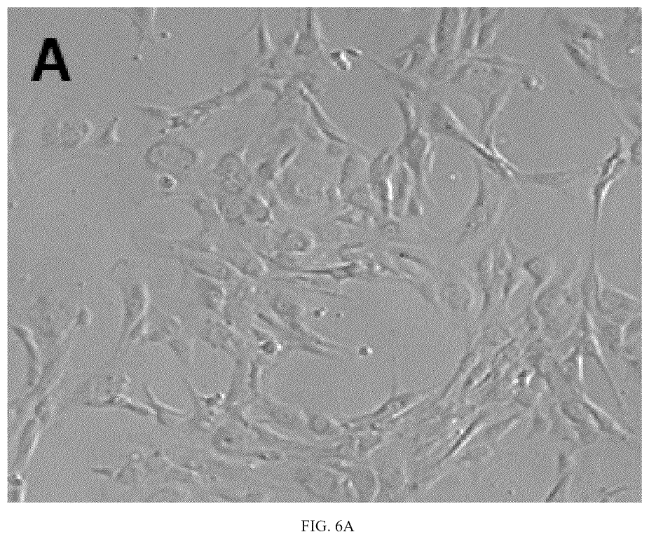

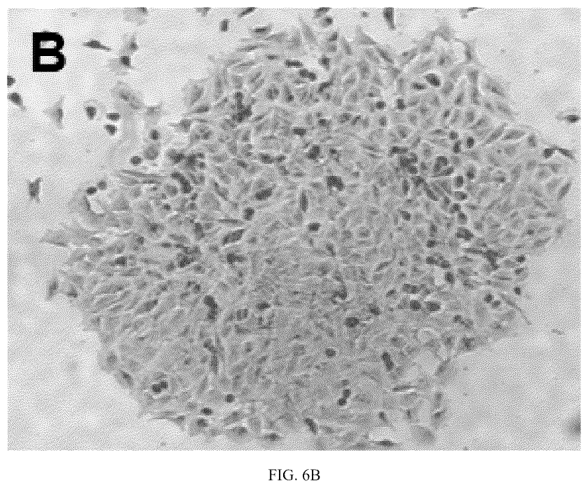

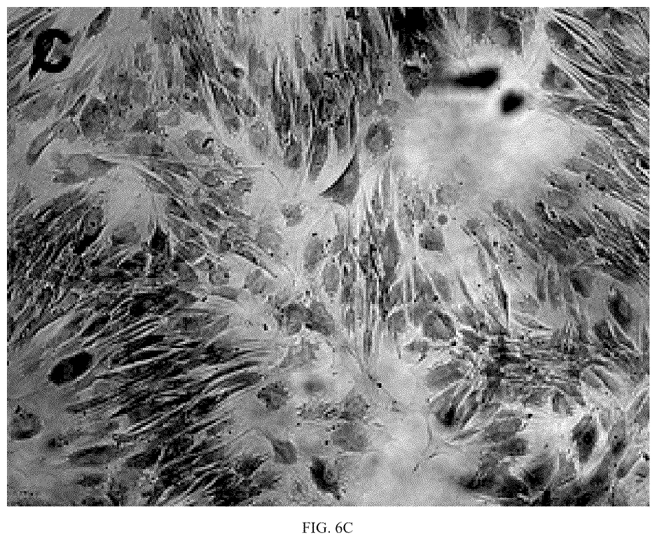

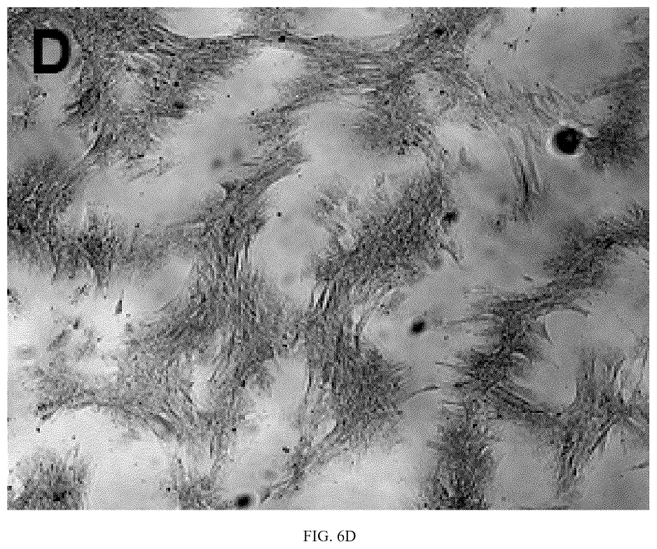

After determining the appropriate cell number and surface, cell migration modeling and differentiation protocol were established using embryoid bodies.
4. 12 ng/ml bFGF was added in it N2B27 medium (DMEMF12/Neurobasal medium (1:1), Bovine Serum Albumin (BSA), N2 supplement, B27 supplement, penicillin (10.000 U/ml)-streptomycin (10.000 μg/ml), L-glutamine) at 4000 cells/well and embryoid bodies were formed using 96-well PCR plates. This process was achieved by precipitating the cells at 200×g for 5 minutes after placing in plastic well culture dishes and then by incubating at 37° C. for 2 days.
5. Embryoid bodies obtained at the end of day two were transferred to 12-well culture dishes coated with Matrigel-gelatin (1:200, 70 μg/ml matrigel-0.1% Gelatin) mixture in the presence of N2B27 medium (DMEMF12/Neurobasal medium (1:1), Bovine Serum Albumin (BSA), N2 supplement, B27 supplement, penicillin (10.000 U/ml)-streptomycin (10.000 μg/ml), L-glutamine) and 3 μM CHIR, and cell migration analyses were performed on day three and day six, and mesoderm marker analyses were performed on day three (FIG. 4).
6. On day three, mesoderm marker analysis was performed in embryoid bodies and mesoderm layer protein expressions were observed (FIGS. 5A-5E).

### Example 4—Mesoderm and Mesenchymal Stem Cell Differentiation

4. Cells were cultured in the presence of CHIR from day two to day six and mesoderm differentiation was completed.
5. As of day six, the medium was replaced with DMEM (1 g/L glucose). 10% FBS and 1% PSA and 5 ng/ml bFGF were added to DMEM and kept in culture until day eight. This process was performed on day six in 10 cm culture dishes coated with Matrigel-gelatin (1:200, 70 μg/ml Matrigel-0.1% Gelatin) mixture. All subsequent differentiation experiments were performed in 10 cm culture dishes coated with Matrigel-gelatin (1:200, 70 mg/ml Matrigel-0.1% Gelatin) mixture until day 24.

As of day eight, cells were kept in DMEM (1 g/L glucose) medium until day 24. 10% FBS and 1% PSA were added to DMEM. Cells at day ten indicate the early mesenchymal stem cell stage and cells at day 24 indicate the adult mesenchymal stem cell stage (FIG. 4).

1. A method for producing mesenchymal stem cells from human pluripotent stem cells, comprising the following steps.
a) forming embryoid bodies from the human pluripotent stem cells;
b) attaching the embryoid bodies to a coated surface to allow a transfer from a 3-dimensional culture to a 2-dimensional culture;
c) inducing the embryoid bodies to allow a differentiation into the mesenchymal stem cells;
d) performing a continuous proliferative culture of the mesenchymal stem cells while maintaining a presence of the mesenchymal stem cells.

2. The method for producing the mesenchymal stem cells according to claim 1, wherein in step a), the human pluripotent stem cells are first precipitated in N2B27 medium supplemented with Rho-kinase inhibitor (ROCKi) and basic fibroblast growth factor (bFGF) at 200×g for 5 minutes and cultured at 37° C. for 2 days to form the embryoid bodies.

3. The method for producing the mesenchymal stem cells according to claim 1, wherein the coated surface in step b) is a surface coated with matrigel-gelatin (1:200, 70 μg/ml matrigel-0.1% Gelatin) to culture 3-dimensional embryoid bodies in a 2-dimensional form and thereby accelerating a process of an epithelial to mesenchymal transition.

4. The method for producing the mesenchymal stem cells according to claim 1, wherein step b) is carried out 2 days after step a).

5. The method for producing the mesenchymal stem cells according to claim 1, wherein the embryoid bodies obtained in step a) are kept for 2 days in step c) and transferred to a surface coated with a matrigel-gelatin mixture in a presence of an N2B27 medium and 3 μM CHIR and cultured in a presence of CHIR for a mesoderm differentiation.

6. The method for producing the mesenchymal stem cells according to claim 1, wherein in step d), the mesenchymal stem cells are cultured in Dulbecco Modified Eagle Medium (DMEM-1 g/L glucose), 10% Fetal bovine serum (FBS), 1% Penicillin-Streptomycin-Amphotericin B (PSA) and 5 ng/ml bFGF and in a dish containing a matrigel-gelatin coated surface.

7. The method for producing the mesenchymal stem cells according to claim 1, wherein early mesenchymal stem cells are formed on day 10 and adult mesenchymal stem cells are formed on day 24.

8. The method for producing the mesenchymal stem cells according to claim 1, wherein the mesenchymal stem cells are configured to differentiate into one of cell groups consisting of adipocytes, osteocytes, chondrocytes, myocytes, neurocytes, and cardiomyocytes.

9. The method for producing the mesenchymal stem cells according to claim 2, wherein the mesenchymal stem cells are configured to differentiate into one of cell groups consisting of adipocytes, osteocytes, chondrocytes, myocytes, neurocytes, and cardiomyocytes.

10. The method for producing the mesenchymal stem cells according to claim 3, wherein the mesenchymal stem cells are configured to differentiate into one of cell groups consisting of adipocytes, osteocytes, chondrocytes, myocytes, neurocytes, and cardiomyocytes.

11. The method for producing the mesenchymal stem cells according to claim 4, wherein the mesenchymal stem cells are configured to differentiate into one of cell groups consisting of adipocytes, osteocytes, chondrocytes, myocytes, neurocytes, and cardiomyocytes.

12. The method for producing the mesenchymal stem cells according to claim 5, wherein the mesenchymal stem cells are configured to differentiate into one of cell groups consisting of adipocytes, osteocytes, chondrocytes, myocytes, neurocytes, and cardiomyocytes.

13. The method for producing the mesenchymal stem cells according to claim 6, wherein the mesenchymal stem cells are configured to differentiate into one of cell groups consisting of adipocytes, osteocytes, chondrocytes, myocytes, neurocytes, and cardiomyocytes.

14. The method for producing the mesenchymal stem cells according to claim 7, wherein the mesenchymal stem cells are configured to differentiate into one of cell groups consisting of adipocytes, osteocytes, chondrocytes, myocytes, neurocytes, and cardiomyocytes.
